(19) United States
(12) Patent Application Publication
(10) Pub. No.: US 2024/0087743 A1
(43) Pub. Date: Mar. 14, 2024

(54) MACHINE LEARNING CLASSIFICATION OF VIDEO FOR DETERMINATION OF MOVEMENT DISORDER SYMPTOMS

(71) Applicant: Videra Health, Inc., Orem, UT (US)

(72) Inventors: Bradley C. Grimm, Riverton, UT (US); Loren D. Larsen, Orem, UT (US); Anthony Alexander Sterns, Akron, OH (US)

(21) Appl. No.: 18/367,389

(22) Filed: Sep. 12, 2023

Related U.S. Application Data
(60) Provisional application No. 63/406,421, filed on Sep. 14, 2022.

(51) Int. Cl. G16H 50/20

(52) U.S. Cl. CPC G16H 50/20

(57) ABSTRACT

A method includes obtaining, by a processing device, video data of a patient, comprising image data and audio data. The method further includes providing, by the processing device, the video data to a first trained machine learning model. The method further include obtaining output from the first trained machine learning model based on the video data, wherein the output includes a first indication that the patient exhibits symptoms of one or more target movement disorders in association with the video data. The method further includes providing an alert to a user indicative of the one or more target movement disorders.

### RELATED APPLICATIONS

This application claims the benefit of U.S. Provisional Application No. 63/406,421, filed Sep. 14, 2022, the entire contents of which are incorporated by reference.

### TECHNICAL FIELD

The present disclosure relates to methods associated with machine learning models used for assessment of movement disorders. Specifically, the present disclosure relates to machine learning models used for classification based on recorded data for determination of movement disorders.

### BACKGROUND

Patients may experience a variety of movement disorders, which may cause unintended, unwanted, and/or involuntary movement or inability of a patient to move one or more body parts as intended. Identification of such movement disorders may be costly in terms of time, expertise, monetary cost, and the like.

### SUMMARY

The following is a simplified summary of the disclosure in order to provide a basic understanding of some aspects of the disclosure. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect of the present disclosure, a method includes obtaining, by a processing device, video data of a patient, comprising image data and audio data. The method further includes providing, by the processing device, the video data to a first trained machine learning model. The method further includes obtaining output from the first trained machine learning model based on the video data, wherein the output includes a first indication that the patient exhibits symptoms of one or more target movement disorders in association with the video data. The method further includes providing an alert to a user indicative of the one or more target movement disorders.

In another aspect of the disclosure, a method includes obtaining a first plurality of video data of a first plurality of patients. The method further includes performing a cropping operation on each of the first plurality of video data to generate a first plurality of video data crops. The method further includes receiving a first plurality of labels associated with each of the first plurality of video data crops. The first plurality of labels comprises a first indication of a presence or absence of evidence of a movement disorder in the first plurality of video data crops. The method further includes training a machine learning model by providing the first plurality of video data crops as training input and the first plurality of labels as target output. The first machine learning model is configured to generate output indicative of whether an input video data crop comprises and indication of the movement disorder.

In another aspect of the disclosure, a non-transitory machine-readable storage medium is disclosed. The storage medium stores instructions which, when executed, cause a processing device to perform operations. The operations include obtaining video data of a patient, including image data and audio data. The operations further include providing the video data to a first trained machine learning model. The operations further include obtaining output from the first trained machine learning model based on the video data. The output includes a first indication that the patient exhibits symptoms of one or more target movement disorders in association with the video data. The operations further include providing an alert to a user indicative of the one or more target movement disorders.

### BRIEF DESCRIPTION OF THE DRAWINGS

The present disclosure is illustrated by way of example, and not by way of limitation in the figures of the accompanying drawings.

FIG. 1 is a block diagram illustrating an exemplary system architecture, according to some embodiments.

FIG. 2 illustrates a model training workflow and a model application workflow, according to some embodiments.

FIG. 3 depicts a frame of a video recording as part of a video for use in determining a presence of a movement disorder, according to some embodiments.

FIG. 4A is a flow diagram of a method for generating a data set for a machine learning model, according to some embodiments.

FIG. 4B is a flow diagram of a method for utilizing machine learning models in determining whether evidence of movement disorders is captured in video data, according to some embodiments.

FIG. 4C is a flow diagram of a method for training a machine learning model for making determinations in association with movement disorders, according to some embodiments.

FIG. 5 depicts an operation flow for making a determination in association with a movement disorder, according to some embodiments.

FIG. 6 is a block diagram illustrating a computer system, according to some embodiments.

### DETAILED DESCRIPTION

Aspects of the present disclosure are directed to utilizing machine learning models to determine if video data of a patient includes evidence of symptoms of one or more movement disorders. Example movement disorders include Huntington's chorea, Parkinson's disease, and tardive dyskinesia (TD). Output of the machine learning models may be utilized in screening patients for further assessment for movement disorders. Output of the machine learning models may be utilized in diagnosing movement disorders. Output of the machine learning models may be utilized in treating movement disorders. Output of the machine learning models may be utilized in determining effectiveness of treatment of movement disorders. Output of the machine learning models may be utilized in improving treatment of movement disorders.

In conventional diagnoses and treatments of movement disorders, a manual screening/assessment may be performed. For example, a physician may perform a test to generate a score indicative of a likelihood that a patient is experiencing symptoms of movement disorders. One such test is the abnormal involuntary movement scale (AIMS) test. Scoring a manual test for movement disorders may be a time-consuming process (e.g., may take between 30 minutes and an hour), may involve special training of the physician, may be influenced by tester bias, etc.

Aspects of the current disclosure enable training and/or utilizing one or more machine learning models to measure and/or determine the presence of movement disorder symptoms from a video of a patient. Aspects of the present disclosure may be applicable to disorders other than TD, symptoms of which are detectable in videos of a patient. In some cases, movement disorders may be intermittent, e.g., involuntary motion or loss of muscle control may occur infrequently, sporadically, randomly, or the like.

In some embodiments, a machine learning model is trained for movement disorders symptom detection. The machine learning model may be trained, validated, selected, tested, etc. The machine learning model may be trained using a training data set, validated using a validation data set, tested using a testing data set, etc. The machine learning model may be a neural network, such as a convolutional neural network. The machine learning model may be a trained machine learning model, e.g., the training dataset may be a labeled data set (e.g., labeled with a target output).

The training data sets (and validating sets and testing sets) may be manually labeled. The data sets may include videos of patients including classification as an output, e.g., whether or not the patient experiences movement disorder symptoms, whether or not movement disorder symptoms are visible in the video, whether or not movement disorder symptoms are detectable in audio, etc. The data sets may be labeled via a scoring system, e.g., related to how many instances of movement disorder symptoms are shown, how severe the patient's movement disorder symptoms are, etc. The data sets may be labeled via a clinical scoring system, e.g., the data sets may be labeled with an AIMS score of the patient or video. The clinical scoring may be performed by a professional, such as a physician.

The training data sets may include videos with portions manually flagged for demonstrating potential movement disorder symptoms. For example, a video may have portions labeled as including involuntary muscle movements. A video may have portions labeled as including audio indicative of movement disorder symptoms. A video may have portions labeled as including speech/language indicative of movement disorders symptoms. Portions labeled may be short, e.g., about one second in duration. Portions labeled may be of any length. Sections of a video that are not labeled as including symptomatic behavior may be separated into portions. For example, the training data set may include a number of portions labeled as including symptomatic behavior, and a number of portions that are not labeled as including symptomatic behavior. The portions not including symptomatic behavior may be separated from the reminder of the video at random intervals. The portions not including symptomatic behavior may be approximately the same length as the portions including symptomatic behavior (e.g., may conform to similar length statistics such as mean, standard deviation, etc., as labeled portions). The portions not including symptomatic behavior may approximately resemble the portions labeled as including symptomatic behavior, e.g., in terms of spread of locations throughout the video from which the portions originate. The portions not including symptomatic behavior may be provided for training, testing, and/or validating meeting a target or threshold proportion of the data provided to the model. For example, the same number of portions including symptomatic behavior and portions not including symptomatic behavior may be provided, twice as many portions not including symptomatic behavior may be provided, etc.

In some embodiments, a training data set (and/or validating and/or testing data set) may include video targeted to certain symptomatic areas. A training video may include a more limited or focused perspective, e.g., videos belonging to a data set may be cropped to a particular symptomatic area. Videos belonging to a data set may be cropped to a body landmark area. For example, training videos may include eyes, brow, face (e.g., muscles of the face), head, jaws, lips, hands, upper extremities, lower extremities, and/or trunk of a patient. Training videos may be cropped to include target areas. For example, a training data set may include videos that have been cropped to include a single symptomatic area. Multiple machine learning models may be trained in association with different symptomatic areas, in some embodiments. A machine learning model associated with a symptomatic region/area may be generated by providing training data that includes video of that symptomatic area.

In some embodiments, a training data set (and/or validating and/or testing data set) may include additional information, such as contextual information. The additional data may provide information that impacts the manifestations of movement disorder symptoms in a patient. Additional data may include any information that affects or is suspected to affect the detectable presence of movement disorder symptoms from the video. Additional data may include medical history, medication history, time of day of the video, etc. For example, additional data may include a history of medications taken by the patient, including medications that may cause movement disorders, medications for treating movement disorders, etc. Additional data may include a historical record of movement disorder symptoms.

In some embodiments, a training data set (and/or validating and/or testing data set) may have noise included, e.g., to generalize results, generalize use of the model, etc. For example, one or more videos of the data sets may be altered, have noise introduced, or the like. Videos may be altered by cropping, rotating, distorting, recoloring, decoloring, blurring, filtering, posterizing, smoothing, or otherwise changing video and/or audio associated with the video of the patent. Introducing noise to the training data sets may decrease reliance of the trained machine learning models on quality of video and/or audio recordings.

In some embodiments, one or more trained machine learning models may be configured to recommend further screening of a patient for movement disorders. One or more trained machine learning models may be configured to generate a likelihood that a patient is exhibiting symptoms of movement disorders. One or more trained machine learning models may be configured to generate a score indicative of a likelihood or severity of movement disorder symptoms. One or more trained learning models may be configured to generate an AIMS score based on one or more videos of a patient.

Results, recommendations, and/or predictions may be generated by providing one or more videos of a patient to one or more machine learning models. Videos may include asking the patient to respond to one or more questions or requests. The questions may include diagnostic questions, such as those asked during an AIMS test. The questions may include open-ended questions, to elicit speech responses. Videos may include recording a patient response to various requests, such as the patient sitting still, standing, walking, positioning their face, lips, jaw, or tongue, tapping their hands, or the like. Videos may include one or more portions of a participant's body, potentially including hands, trunk, face, eyes, jaw, tongue, lips, etc. Videos may include one or more target portions of a patient's body.

One or more videos may be provided to one or more trained machine learning models. The one or more trained machine learning models may perform one or more of a number of operations. Operations performed by trained machine learning models may include partitioning a video. Partitioning may include spatial partitioning. For example, portions of a video may be cropped to highlight or accentuate a particular body part, symptomatic region, or the like. Video recording data may be cropped to include one or more target portions of a patient's body. Partitioning may include temporal partition. For example, portions of a video that include a target body part may be separated from portions that do not include the body part. Portions of a video may be provided to a corresponding machine learning model. For example, portions of a video that have been cropped temporally and/or spatially for a target body part may be provided to a machine learning model associated with the target body part. Multiple temporal and/or spatial portions of a video may be provided to different trained machine learning models associated with different body parts, body landmark regions, or the like.

Operations performed by trained machine learning models may include detecting portions of a video that include evidence of movement disorder symptoms. A machine learning model may detect involuntary motions. The machine learning model may detect motion of the patient that is indicative of one or more movement disorders. The machine learning model may separate, flag, or the like portions of the video that include motion indicative of movement disorders. Operations performed by the trained machine learning models may further include separating portions of a video that do not include evidence of movement disorder symptoms. A machine learning model may utilize both video data with predicted movement disorder symptoms depicted and video data without predicted movement disorder symptoms, e.g., to provide a baseline or comparison.

Operations performed by trained machine learning models may include determining whether a video indicates that a patient is experiencing symptoms of movement disorders. A machine learning model may be provided with video data to determine whether the video is indicative of movement disorders. The machine learning model may be provided portions of a video, e.g., cropped to include one or more target body parts or target portions of a patient's body, portions of a video flagged as including evidence of movement disorders, portions of a video flagged as not including evidence of movement disorders, etc. The machine learning model may be provided with additional information that may contribute to a prediction/recommendation. For example, the model may be provided with additional information that may affect presentation of movement disorder symptoms, such as medical history, medication history, etc. The model may use the additional information to increase an accuracy of predictions, accuracy of recommendations, or the like.

Operations performed by the trained machine learning models may include determining a risk, classification, and/or score of a patient experiencing movement disorder symptoms. Determining a prediction of risk, a classification of symptoms, or a score associated with a movement disorder may include receiving output from one or more models. For example, a machine learning model (e.g., a fusion model for analyzing output of several other models) may collect data from models associated with different body parts, models associated with audio, and/or models associated with speech, and utilize that body of machine learning output to make a prediction about a patent's risk of experiencing movement disorder symptoms. Operations performed by the trained machine learning models may include providing a classification, such as whether or not to recommend further screening. Operations performed by the trained machine learning models may include providing a risk factor, such as a likelihood that the patient is experiencing movement disorder symptoms. Operations performed by the trained machine learning models may include providing a score, such as a predicted AIMS score.

Operations for screening, diagnosing, and/or treating patients for movement disorders may include storing historical data, accessing historical data, processing historical data, etc. For example, a patient's AIMS score as determined by methods and systems of the present disclosure may be tracked over time to determine a progressive movement disorder severity, an effectiveness of a treatment plan, etc.

Operations of the present disclosure may be performed by a model, such as a trained machine learning model. Operations of the present disclosure may be performed by multiple models. Operations of the present disclosure may be performed by multiple models working together, e.g., an ensemble model.

Operations of the present disclosure may be utilized as screening for movement disorder symptoms, e.g., they may be utilized for recommending further investigations into movement disorder symptoms exhibited by a patient. Operations of the present disclosure may be utilized as diagnostic tools for movement disorders. Operations of the present disclosure may be utilized as a treatment tool, e.g., in evaluating treatment effectiveness for a movement disorder treatment plan.

In one aspect of the present disclosure, a method includes obtaining, by a processing device, video data of a patient, comprising image data and audio data. The method further includes providing, by the processing device, the video data to a first trained machine learning model. The method further includes obtaining output from the first trained machine learning model based on the video data, wherein the output includes a first indication that the patient exhibits symptoms of one or more target movement disorders in association with the video data. The method further includes providing an alert to a user indicative of the one or more target movement disorders.

In another aspect of the disclosure, a method includes obtaining a first plurality of video data of a first plurality of patients. The method further includes performing cropping of each of the first plurality of video data to generate a first plurality of video data crops. The method further includes receiving a first plurality of labels associated with each of the first plurality of video data crops. The first plurality of labels comprises a first indication of a presence or absence of evidence of a movement disorder in the first plurality of video data crops. The method further includes training a machine learning model by providing the first plurality of video data crops as training input and the first plurality of labels as target output. The first machine learning model is configured to generate output indicative of whether an input video data crop comprises and indication of the movement disorder.

In another aspect of the disclosure, a non-transitory machine-readable storage medium is disclosed. The storage medium stores instructions which, when executed, cause a processing device to perform operations. The operations include obtaining video data of a patient, including image data and audio data. The operations further include providing the video data to a first trained machine learning model. The operations further include obtaining output from the first trained machine learning model based on the video data. The output includes a first indication that the patient exhibits symptoms of one or more target movement disorders in association with the video data. The operations further include providing an alert to a user indicative of the one or more target movement disorders.

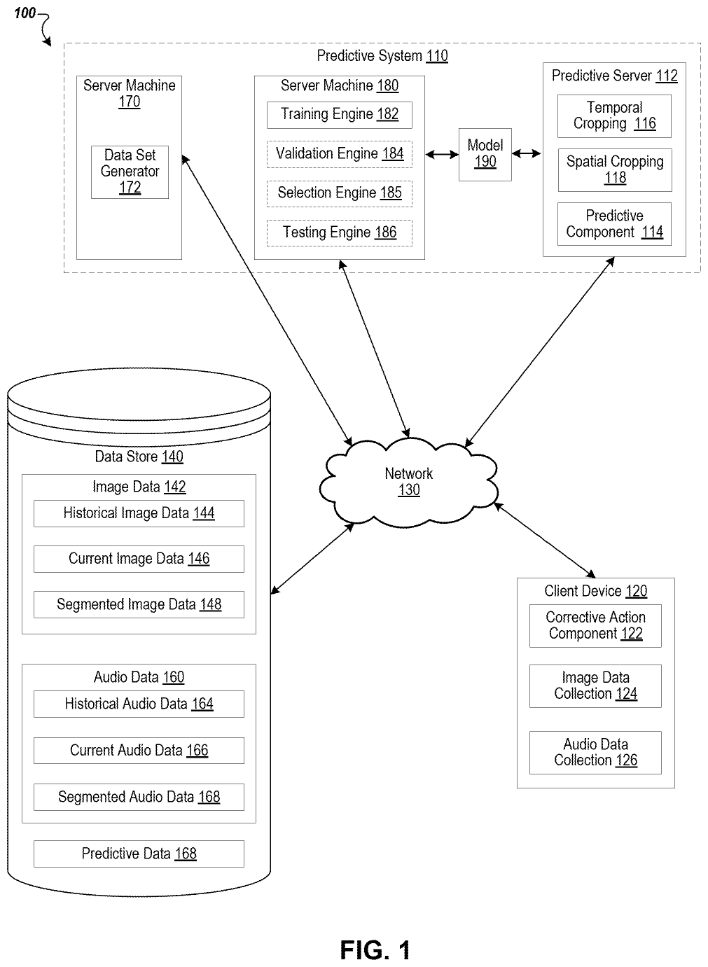

FIG. 1 is a block diagram illustrating an exemplary system 100 (exemplary system architecture), according to some embodiments. The system 100 includes a client device 120, a predictive server 112, and a data store 140. The predictive server 112 may be part of predictive system 110. Predictive system 110 may further include server machines 170 and 180.

Client device 120 includes apparatuses for image data collection 124 and audio data collection 126. For example, client device 120 may include a camera and a microphone. Client device 120 may be configured to generate video data based on image data collection 124 and audio data collection 126. Client device 120 may be utilized to collect video data of a patient, e.g., a patient who may be exhibiting symptoms of a movement disorder.

Image data collection 124 and audio data collection 126 may be utilized in collecting image data 142 and audio data 160. Image data 142 and audio data 160 may together comprise video recording data of patients, e.g., patients who may be exhibiting movement disorder symptoms. Video recording data of patients may include historical recordings and current recordings. Video recording data of patients may include recordings generated to be provided for methods performed by system 100, or recordings for other purposes provided to system 100. For example, a virtual appointment with a health care provider may be recorded for further operations to be performed by one or more components of system 100. In a further example, a movement disorder assessment may be recorded, such as an assessment including prompting speech and movement patterns for determining whether a patient is experiencing a movement disorder.

Historical image data 144 may be associated with historical data collection, such as historical video recordings. Historical image data 144 may include a series of image frames of patients. Historical image data 144 may be utilized in configuring a machine learning model to perform one or more tasks. For example, historical image data 144 may be used for training, validating, testing, etc., the machine learning model. Historical image data 144 may be provided to a machine learning model to adjust parameters of one or more model parameters that enable learning of the machine learning model. Historical image data 144 may be labeled, e.g., may be classified according to whether or not evidence of movement disorders may be observed in a series of images, e.g., a video clip. Historical audio data 164 may share one or more features with historical image data 144. A portion of historical audio data 164 and historical image data 144 may together form a historical video clip.

Current image data 146 may be image data associated with a recording of a patient to be screened for a movement disorder. Current image data 146 may be utilized as input to a trained machine learning model. Current audio data 166 may share one or more features with current image data 146. Segmented image data 148 may include spatially and/or temporally cropped images, series of images, video clips, or the like. For example, output of spatial cropping 118 or temporal cropping 116 may be or include segmented image data 148. Segmented image data 148 may include historical and/or current image data. Segmented image data 148 may be utilized in configuring a machine learning model, provided as input to a trained machine learning model, etc. Segmented audio data 168 may share one or more features with segmented image data 148. Segmented audio data 168 may also be provided as input to a trained machine learning model. In some embodiments, audio data and corresponding image data (e.g., video data) may be provided as input to one or more trained machine learning models.

In some embodiments, image data 142 and/or audio data 160 may be processed (e.g., by the client device 120 and/or by the predictive server 112). Processing of the sensor data 142 may include generating features. In some embodiments, the features are a pattern in the image data 142 and/or audio data 160 (e.g., slope, width, height, peak, etc.) or a combination of values from the image data 142 and audio data 160 (e.g., composite video data, etc.). In some embodiments, processed data may be cropped data, spatially and/or temporally. Processed data may be used for performing signal processing and/or for obtaining predictive data 168 for performance of a corrective action.

Each instance (e.g., set) of image data 142 and audio data 160 may correspond to a crop or segment of a video recording, a video recording, a patient (e.g., including multiple recordings), or the like. A set of image data 142 and audio data 160 may comprise video recording data. The data store may further store information associating sets of different data types, e.g., information indicative that a set of image data 142 and a set of audio data 160 are associated with the same patient, or the like.

In some embodiments, predictive system 110 may generate predictive data 168 using supervised machine learning. For example, predictive data 168 may include output from a machine learning model that was trained using labeled data, such as video data labeled with a diagnosis of a movement disorder of a subject of the video data. In some embodiments, predictive system 110 may generate predictive data 168 using unsupervised machine learning (e.g., predictive data 168 includes output from a machine learning model that was trained using unlabeled data, output may include clustering results, principle component analysis, anomaly detection, etc.). In some embodiments, predictive system 110 may generate predictive data 168 using semi-supervised learning (e.g., training data may include a mix of labeled and unlabeled data, etc.).

Client device 120, predictive server 112, data store 140, server machine 170, and server machine 180 may be coupled to each other via network 130 for generating predictive data 168 to perform corrective actions. In some embodiments, network 130 may provide access to cloud-based services. Operations performed by client device 120, predictive system 110, data store 140, etc., may be performed by virtual cloud-based devices.

In some embodiments, network 130 is a public network that provides client device 120 with access to the predictive server 112, data store 140, and other publicly available computing devices. In some embodiments, network 130 is a private network that provides client device 120 access to privately available computing devices, such as data store 140, additional client devices (not shown), etc. Network 130 may include one or more Wide Area Networks (WANs), Local Area Networks (LANs), wired networks (e.g., Ethernet network), wireless networks (e.g., an 802.11 network or a Wi-Fi network), cellular networks (e.g., a Long Term Evolution (LTE) network), routers, hubs, switches, server computers, cloud computing networks, and/or a combination thereof.

Client device 120 may include computing devices such as Personal Computers (PCs), laptops, mobile phones, smart phones, tablet computers, netbook computers, network-connected televisions (“smart TV”), network-connected media players (e.g., Blu-ray players), set-top-boxes, Over-the-Top (OTT) streaming devices, operator boxes, etc. Client device 120 may include a corrective action component 122. Corrective action component 122 may receive user input (e.g., via a Graphical User Interface (GUI) displayed via the client device 120) of an indication associated with determining whether a movement disorder is present in video data. In some embodiments, corrective action component 122 transmits the indication to the predictive system 110, receives output (e.g., predictive data 168) from the predictive system 110, determines a corrective action based on the output, and causes the corrective action to be implemented. In some embodiments, corrective action component 122 obtains data (e.g., current image data 146 from data store 140, etc.) and provides data associated with one or more movement disorder patients to predictive system 110.

In some embodiments, corrective action component 122 receives an indication of a corrective action from the predictive system 110 and causes the corrective action to be implemented. Each client device 120 may include an operating system that allows users to one or more of generate, view, or edit data (e.g., indications associated with one or more patients, corrective actions associated with one or more patients, etc.).

In some embodiments, the corrective action includes providing an alert to a user. For example, client device 120 may be caused to display an alert indicating to a healthcare provider that a patient has exhibited signs of one or more movement disorders. Client device 120 may indicate that a subject of one or more video data has a probability above a threshold probability of experiencing tardive dyskinesia, Parkinson's disease, Huntington's chorea, or another movement disorder. In some embodiments, a machine learning model is trained to monitor a recording of a patient, e.g., including image and audio data. In some embodiments, the machine learning model may generate output indicative of a likelihood that a clip, recording, or patient is exhibiting signs of one or more target movement disorders.

Predictive server 112, server machine 170, and server machine 180 may each include one or more computing devices such as a rackmount server, a router computer, a server computer, a personal computer, a mainframe computer, a laptop computer, a tablet computer, a desktop computer, Graphics Processing Unit (GPU), accelerator Application-Specific Integrated Circuit (ASIC) (e.g., Tensor Processing Unit (TPU)), etc. Operations of predictive server 112, server machine 170, server machine 180, data store 140, etc., may be performed by a cloud computing service, cloud data storage service, etc.

Predictive server 112 may include a predictive component 114. In some embodiments, the predictive component 114 may receive current image data 146, and/or current audio data 166 (e.g., receive from the client device 120, retrieve from the data store 140) and generate output (e.g., predictive data 168) for performing corrective action associated with one or more patients based on the current data. In some embodiments, predictive data 168 may include one or more predicted likelihoods that a clip, video recording, or patient is exhibiting symptoms of one or more target movement disorders. In some embodiments, predictive data 168 may indicate a severity of one or more target movement disorders exhibited in a clip, video recording, or by a patient. Predictive component 114 may utilize one or more trained machine learning models 190 to determine the output for performing the corrective action based on the current data.

One type of machine learning model that may be used to perform some or all of the above tasks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top-layer features extracted by the convolutional layers to decisions (e.g., classification outputs).

A recurrent neural network (RNN) is another type of machine learning model. A recurrent neural network model is designed to interpret a series of inputs where inputs are intrinsically related to one another, e.g., time trace data, sequential data, etc. Output of a perceptron of an RNN is fed back into the perceptron as input, to generate the next output.

A transformer architecture is another type of machine learning model that may be utilized in connection with the present disclosure. A transformer included an attention mechanism, which allows it to capture relationships between various portions of an input without relying on recurrent layers. Transformers add positional encodings to various portions of input data to enable the attention mechanism to determine correlations and importance of correlations between the various portions of input data. The attention mechanism amplifies importance of some portions of input data while suppressing signal from unimportant input data.

Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher-level shapes (e.g., teeth, lips, gums, etc.); and the fourth layer may recognize a scanning role. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

In some embodiments, predictive component 114 receives current image data 146 and/or current audio data 166, performs signal processing to break down the current data into sets of current data, provides the sets of current data as input to a trained model 190, and obtains outputs indicative of predictive data 168 from the trained model 190. In some embodiments, predictive server 112 may receive current data (e.g., video data including image data and audio data), provide the current data to temporal cropping 116 to generate clips and/or spatial cropping 118 to crop images to target body parts, and provided segmented image data 148 and/or segmented audio data 168 (e.g., segmented by spatial cropping 118 and/or temporal cropping 116) to model 190 for generating predictive data.

In some embodiments, the various models discussed in connection with model 190 (e.g., supervised machine learning model, unsupervised machine learning model, etc.) may be combined in one model (e.g., an ensemble model), or may be separate models.

Data may be passed back and forth between several distinct models included in model 190 and predictive component 114. In some embodiments, some or all of these operations may instead be performed by a different device, e.g., client device 120, server machine 170, server machine 180, etc. It will be understood by one of ordinary skill in the art that variations in data flow, which components perform which processes, which models are provided with which data, and the like are within the scope of this disclosure.

Data store 140 may be a memory (e.g., random access memory), a drive (e.g., a hard drive, a flash drive), a database system, a cloud-accessible memory system, or another type of component or device capable of storing data. Data store 140 may include multiple storage components (e.g., multiple drives or multiple databases) that may span multiple computing devices (e.g., multiple server computers). The data store 140 may store image data 142, audio data 160 (e.g., video data), and predictive data 168.

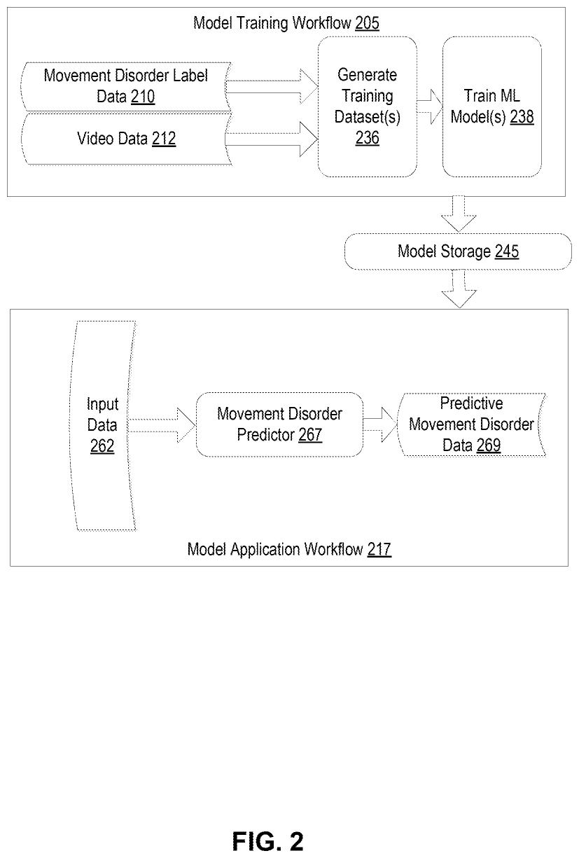

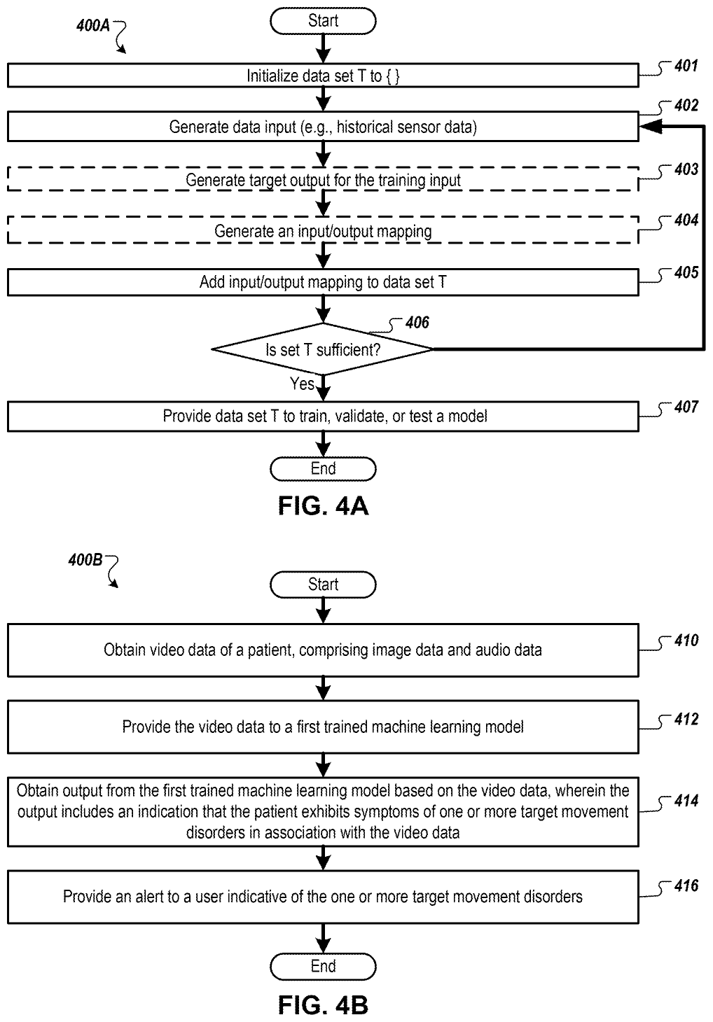

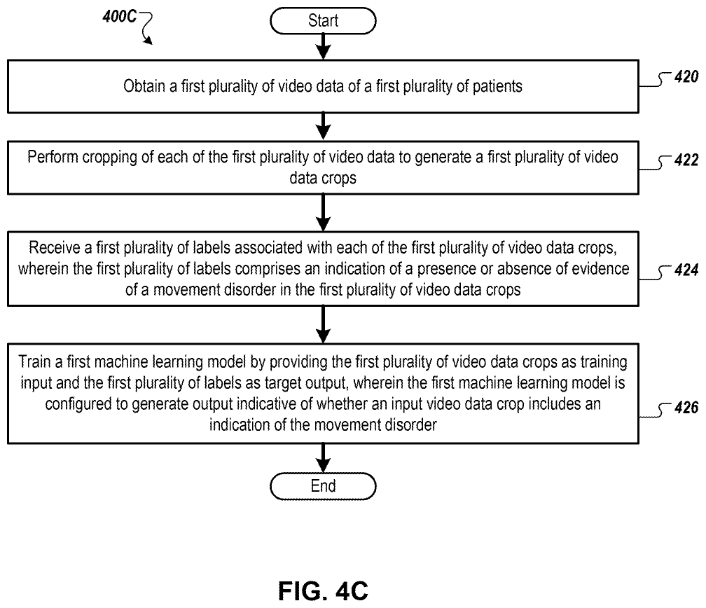

In some embodiments, predictive system 110 further includes server machine 170 and server machine 180. Server machine 170 includes a data set generator 172 that is capable of generating data sets (e.g., a set of data inputs and a set of target outputs) to train, validate, and/or test model(s) 190, including one or more machine learning models. Some operations of data set generator 172 are described in detail below with respect to FIGS. 2 and 4A. In some embodiments, data set generator 172 may partition the historical data (e.g., historical image data 144, historical audio data 164) into a training set (e.g., sixty percent of the historical data), a validating set (e.g., twenty percent of the historical data), and a testing set (e.g., twenty percent of the historical data).

In some embodiments, predictive system 110 (e.g., via predictive component 114) generates multiple sets of features. For example, a first set of features may correspond to a first set of crops of video data (e.g., a first sequence of video frames stored included in image data, a first spatial crop targeting a specific portions of the patient's body, or the like) that correspond to each of the data sets (e.g., training set, validation set, and testing set), and a second set of features may correspond to a second set of crops of video data (e.g., from temporal and/or spatial cropping) that correspond to each of the data sets. The video data may include audio data, image data (e.g., sequences of images), etc. In some embodiments, a machine learning model may receive as input, output from one or more other machine learning models. For example, one or more models may provide information indicative of evidence of movement disorders in one or more clips of a video, a second model may receive output of the first one or more models and provide output indicative of a likelihood the video as a whole presents evidence of a movement disorder, and a third model may receive output of the second model in association with several recorded videos and determine a likelihood, based on the videos, that a patient is experiencing symptoms of one or more movement disorders.

Server machine 180 includes a training engine 182, a validation engine 184, selection engine 185, and/or a testing engine 186. An engine (e.g., training engine 182, a validation engine 184, selection engine 185, and a testing engine 186) may refer to hardware (e.g., circuitry, dedicated logic, programmable logic, microcode, processing device, etc.), software (such as instructions run on a processing device, a general purpose computer system, or a dedicated machine), firmware, microcode, or a combination thereof. The training engine 182 may be capable of training a model 190 using one or more sets of features associated with the training set from data set generator 172. The training engine 182 may generate multiple trained models 190, where each trained model 190 corresponds to a distinct set of features of the training set (e.g., a different collection of video crops, output of a classification model, etc.). For example, a first trained model may have been trained using all features (e.g., X1-X5), a second trained model may have been trained using a first subset of the features (e.g., X1, X2, X4), and a third trained model may have been trained using a second subset of the features (e.g., X1, X3, X4, and X5) that may partially overlap the first subset of features. Data set generator 172 may receive the output of a trained model, collect that data into training, validation, and testing data sets, and use the data sets to train a second model (e.g., a machine learning model configured to output predictive data, corrective actions, etc.).

Validation engine 184 may be capable of validating a trained model 190 using a corresponding set of features of the validation set from data set generator 172. For example, a first trained machine learning model 190 that was trained using a first set of features of the training set may be validated using the first set of features of the validation set. The validation engine 184 may determine an accuracy of each of the trained models 190 based on the corresponding sets of features of the validation set. Validation engine 184 may discard trained models 190 that have an accuracy that does not meet a threshold accuracy. In some embodiments, selection engine 185 may be capable of selecting one or more trained models 190 that have an accuracy that meets a threshold accuracy. In some embodiments, selection engine 185 may be capable of selecting the trained model 190 that has the highest accuracy of the trained models 190.

Testing engine 186 may be capable of testing a trained model 190 using a corresponding set of features of a testing set from data set generator 172. For example, a first trained machine learning model 190 that was trained using a first set of features of the training set may be tested using the first set of features of the testing set. Testing engine 186 may determine a trained model 190 that has the highest accuracy of all of the trained models based on the testing sets.

In the case of a machine learning model, model 190 may refer to the model artifact that is created by training engine 182 using a training set that includes data inputs and corresponding target outputs (correct answers for respective training inputs). Patterns in the data sets can be found that map the data input to the target output (the correct answer), and machine learning model 190 is provided mappings that capture these patterns. The machine learning model 190 may use one or more of Support Vector Machine (SVM), Radial Basis Function (RBF), clustering, supervised machine learning, semi-supervised machine learning, unsupervised machine learning, k-Nearest Neighbor algorithm (k-NN), linear regression, random forest, neural network (e.g., artificial neural network, recurrent neural network), etc.

In some embodiments, one or more machine learning models 190 may be trained using historical data (e.g., historical image data 144, historical audio data 166, potentially segmented either spatially and/or temporally).

Predictive component 114 may provide current data to model 190 and may run model 190 on the input to obtain one or more outputs. For example, predictive component 114 may provide current image data 146 to model 190 and may run model 190 on the input to obtain one or more outputs. Predictive component 114 may be capable of determining (e.g., extracting) predictive data 168 from the output of model 190. Predictive component 114 may determine (e.g., extract) confidence data from the output that indicates a level of confidence that predictive data 168 is an accurate predictor of a process associated with the input data for patients that may be experiencing movement disorders. Predictive component 114 or corrective action component 122 may use the confidence data to decide whether to cause a corrective action based on predictive data 168.

The confidence data may include or indicate a level of confidence that the predictive data 168 is an accurate prediction of movement disorders associated with at least a portion of the input data. In one example, the level of confidence is a real number between 0 and 1 inclusive, where 0 indicates no confidence that the predictive data 168 is an accurate prediction for one or more movement disorders according to input data, and 1 indicates absolute confidence that the predictive data 168 accurately predicts one or more movement disorders according to input data. Responsive to the confidence data indicating a level of confidence below a threshold level for a predetermined number of instances (e.g., percentage of instances, frequency of instances, total number of instances, etc.) predictive component 114 may cause trained model 190 to be re-trained (e.g., based on current image data 146, current audio data 166, etc.). In some embodiments, retraining may include generating one or more additional data sets (e.g., via data set generator 172) utilizing historical data.

Performing comprehensive screening for movement disorders can be expensive, convenient, training-dependent, and unreliable. For example, screening for tardive dyskinesia may require a trained professional to have a scheduled meeting with a potential patient, and may require the professional to keenly observe the patient during the scheduled meeting. In addition, an appointment with a health care provider who is not an expert in one or more movement disorders may not be sufficient to screen for or diagnose a movement disorder. By providing video data (e.g., from a routine or scheduled virtual appointment with any healthcare provider, or video provided by a patient for screening) to a trained machine learning model, and receiving output indicative of a likelihood of the patient exhibiting symptoms of a movement disorder, diagnoses may be more quickly delivered, referrals to specialists may be made, and treatment may be begun earlier, which may improve the quality of life of a patient, without requiring the patient to have regular meetings with a professional trained in recognizing one or more movement disorders.

For purpose of illustration, rather than limitation, aspects of the disclosure describe the training of one or more machine learning models 190 using historical data to determine predictive data 168. In other embodiments, a heuristic model, physics-based model, or rule-based model is used to determine predictive data 168 (e.g., without using a trained machine learning model). In some embodiments, such models may be trained using historical data. In some embodiments, these models may be retrained utilizing a combination of historical data current data. Any of the information described with respect to data inputs 262 of FIG. 2 may be monitored or otherwise used in the heuristic, physics-based, or rule-based model.

In some embodiments, the functions of client device 120, predictive server 112, server machine 170, and server machine 180 may be provided by a fewer number of machines. For example, in some embodiments server machines 170 and 180 may be integrated into a single machine, while in some other embodiments, server machine 170, server machine 180, and predictive server 112 may be integrated into a single machine. In some embodiments, client device 120 and predictive server 112 may be integrated into a single machine. In some embodiments, functions of client device 120, predictive server 112, server machine 170, server machine 180, and data store 140 may be performed by a cloud-based service.

In general, functions described in one embodiment as being performed by client device 120, predictive server 112, server machine 170, and server machine 180 can also be performed on predictive server 112 in other embodiments, if appropriate. In addition, the functionality attributed to a particular component can be performed by different or multiple components operating together. For example, in some embodiments, the predictive server 112 may determine the corrective action based on the predictive data 168. In another example, client device 120 may determine the predictive data 168 based on output from the trained machine learning model.

In addition, the functions of a particular component can be performed by different or multiple components operating together. One or more of the predictive server 112, server machine 170, or server machine 180 may be accessed as a service provided to other systems or devices through appropriate application programming interfaces (API).

Further, in some embodiments, multiple devices may perform actions ascribed to a single component. For example, a patient may record a video on their own device (e.g., including image data collection 124 and audio data collection 126), and a healthcare provider's device may generate one or more alerts for a user (e.g., via corrective action component 122).

In embodiments, a “user” may be represented as a single individual. However, other embodiments of the disclosure encompass a “user” being an entity controlled by a plurality of users and/or an automated source. For example, a set of individual users federated as a group of administrators may be considered a “user.”

FIG. 2 illustrates a model training workflow 205 and a model application workflow 217 for movement disorder determination, in accordance with some embodiments of the present disclosure. In embodiments, the model training workflow 205 may be performed at a server which may or may not include a movement disorder predictive data generation application, and the trained models are provided to a predictive component (e.g., on predictive server 112 of FIG. 1), which may perform the model application workflow 217. The model training workflow 205 and the model application workflow 217 may be performed by processing logic executed by a processor of a computing device. One or more of these workflows 205, 217 may be implemented, for example, by one or more machine learning modules implemented by server 112 of FIG. 1.

In some embodiments, the trained machine learning model is a neural network, a decision tree, a random forest model, a support vector machine, or other types of machine learning models.

In some embodiments, the trained machine learning model is an artificial neural network (also referred to simply as a neural network). The artificial neural network may be, for example, a convolutional neural network (CNN) or a deep neural network. In some embodiments, processing logic performs supervised machine learning to train the neural network.

Artificial neural networks generally include a feature representation component with a classifier or regression layers that map features to a target output space. A convolutional neural network (CNN), for example, hosts multiple layers of convolutional filters. Pooling is performed, and non-linearities may be addressed, at lower layers, on top of which a multi-layer perceptron is commonly appended, mapping top-layer features extracted by the convolutional layers to decisions (e.g., classification outputs). The neural network may be a deep network with multiple hidden layers or a shallow network with zero or a few (e.g., 1-2) hidden layers. Deep learning is a class of machine learning algorithms that use a cascade of multiple layers of nonlinear processing units for feature extraction and transformation. Each successive layer uses the output from the previous layer as input. Neural networks may learn in a supervised (e.g., classification) and/or unsupervised (e.g., pattern analysis) manner. Some neural networks (e.g., such as deep neural networks) include a hierarchy of layers, where the different layers learn different levels of representations that correspond to different levels of abstraction. In deep learning, each level learns to transform its input data into a slightly more abstract and composite representation.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

The trained machine learning model may be periodically or continuously retrained to achieve continuous learning and improvement of the trained machine learning model. The model may generate an output based on an input, an action may be performed based on the output, and a result of the action may be measured. In some instances, the result of the action is measured within seconds or minutes, and in some instances, it takes longer to measure the result of the action. For example, one or more additional processes may be performed before a result of the action can be measured. The action and the result of the action may indicate whether the output was a correct output and/or a difference between what the output should have been and what the output was. Accordingly, the action and the result of the action may be used to determine a target output that can be used as a label for the sensor measurements. Once the result of the action is determined, the input to the model (e.g., one or more video clips of a patient), the output of the trained machine learning model (e.g., a prediction of movement disorders), and the target result (e.g., recommended corrective actions) and actual measured result (e.g., further determination based on an evaluation by a heath care expert of the presence of a movement disorder in the patient) may be used to generate a new training data item. The new training data item may then be used to further train the trained machine learning model. This retraining process may be performed by computing devices that performed the initial training and/or configuring of the model, or by one or more different computing devices.

The model training workflow 205 is to train one or more machine learning models (e.g., deep learning models) to perform one or more classifying, segmenting, detection, recognition, decision, etc. tasks associated with predicting one or more movement disorders. The model application workflow 217 is to apply the one or more trained machine learning models to perform the classifying, segmenting, detection, recognition, determining, etc., tasks for identifying symptoms of movement disorders from input data. One or more of the machine learning models may receive and process result data (e.g., evaluations of patients by health care providers) and input data (e.g., recorded video data).

Various machine learning outputs are described herein. Particular numbers and arrangements of machine learning models are described and shown. However, it should be understood that the number and type of machine learning models that are used and the arrangement of such machine learning models can be modified to achieve the same or similar end results. Accordingly, the arrangements of machine learning models that are described and shown are merely examples and should not be construed as limiting.

In embodiments, one or more machine learning models are trained to perform one or more of the below tasks. Each task may be performed by a separate machine learning model. Alternatively, a single machine learning model (e.g., an ensemble model) may perform all of the tasks or a subset of the tasks. Additionally, or alternatively, different machine learning models may be trained to perform different combinations of the tasks. In an example, one or a few machine learning models may be trained, where the trained machine learning model is a single shared neural network that has multiple shared layers and multiple higher-level distinct output layers, where each of the output layers outputs a different prediction, classification, identification, etc. The tasks that the one or more trained machine learning models may be trained to perform are as follows:
1. Cropping of video data—Recorded or live video of a patient may be cropped (e.g., temporally and/or spatially) for further processing. Temporal cropping may be performed to generate shorter time clips than an entire video recording of a patient. Clips may be generated that approximately correspond to expected lengths of symptomatic display of movement disorders. For example, image frames of a patient being screened for tardive dyskinesia may be separated into approximately one-second periods, which may approximately correspond to an involuntary movement pattern caused by tardive dyskinesia. Clips of any temporal length may be generated, based on intended usage, for example, about one second, 0.5 to 5 seconds, 0.1 to 10 seconds, or other lengths may be used. Various temporal crops (potentially temporally overlapping) may be taken from a single video recording, which may be of uniform or variable length. In another example, audio of a patient may be cropped temporally into segments approximately corresponding to expected length of an episode of a movement disorder symptom that is detectable in patient speech. Spatial cropping may be performed on image data (e.g., video frames) to highlight, accentuate, or zoom in on portions of a body expected to demonstrate one or more movement disorders. For example, tardive dyskinesia is particularly likely to be demonstrated in movements of the jaw, lips, tongue, eyes, muscles of the face, and hands. One or more machine learning models may be utilized to generate cropped videos focusing on a target body location. In some embodiments, a large number of crops of each video recording of a patient may be provided to various machine learning models. Clips may be cropped both temporally and spatially. For example, a video clip may be generated for further processing that is approximately one second long, and focuses on the jaw of a patient.
2. Determination of movement disorder symptoms based on video data—One or more machine learning models may be trained to determine whether a movement disorder was exhibited in a series of image frames and/or audio of recorded video data. The one or more machine learning models trained to determine the presence of movement disorder symptoms may be provided with cropped (spatially and/or temporally) video data. The one or more machine learning models trained to determine the presence of movement disorder symptoms may be provided with many crops of the video data, e.g., many approximately one-second clips, many spatial crops focusing on particular body parts, or the like. The one or more machine learning models trained to determine the presence of movement disorder symptoms may output a likelihood that a clip includes a demonstration of a movement disorder. The one or more machine learning models trained to determine the presence of movement disorder symptoms may output a projection into a neural space (for example, a penultimate layer of a conventional classification network) that may include additional information represented in an N-dimensional vector space.
3. Determination of a movement disorder exhibited in a video recording—one or more machine learning models may be trained to determine, based on output of one or more machine learning models that operate on various data crops, a likelihood that an entire video is indicative of one or more target movement disorders. A machine learning model may receive, as input, output from one or more other machine learning models, such as those trained to determine movement disorder symptoms based on video data described in task two, above. The machine learning model may receive an N-dimensional embedding. For example, a final layer of the machine learning model may be a classifier layer, with a number of output nodes dependent upon the number of possible classifications. A layer of nodes before the final classification layer may include more nodes than the classification layer, and may carry some additional information, encoded as a vector value with dimensionality equal to the number of nodes of the layer. The N-dimensional embedding provided to the machine learning model may be values of nodes of a layer of a separate machine learning classifier before the classification (e.g., conventional output) layer. The machine learning model may determine, based on the data provided based on a variety of temporal and/or spatial crops of a video, whether the video as a whole indicates the demonstration of one or more movement disorders. The machine learning model may determine a composite indication for the video based on determinations of a number of crops of the video. Likelihood of a disorder, severity of a disorder, etc., may further be predicted. The model may be configured to predict severity of the disorder by providing labels indicative of a severity of the disorder in patients along with the video data of the associated patients.
4. Determination of a movement disorder presenting in a patient—one or more machine learning models may be configured to determine, based on multiple video samples, whether a patient is experiencing a movement disorder. The machine learning model may receive, as input, output from one or more other machine learning models, such as those trained to determine movement disorders exhibited in a video recording described in task three above. The machine learning model may generate a composite indication for the patient based on machine learning output determined from multiple videos. The machine learning model may track progress of symptoms, disorders, and/or treatments over time by being provided with dated information associated with one or more video data. The machine learning model may predict severity of symptoms of the movement disorder. The machine learning model may be trained to predict severity of symptoms by being provided with training data of patients along with symptom severity labels of the associated patients. The machine learning model may receive an N-dimensional embedding as input, e.g., a layer of a machine learning classifier before the classification layer.

One type of machine learning model that may be used to perform some or all of the above tasks is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top-layer features extracted by the convolutional layers to decisions (e.g., classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset.

For the model training workflow 205, a training dataset containing hundreds, thousands, tens of thousands, hundreds of thousands or more of movement disorder label data 210 (e.g., labels to historical video data provided by trained experts) may be used to form a training dataset. In embodiments, the training dataset may also include associated video data 212 for forming a training dataset, where each data point and/or associated recombination configuration may include various labels or classifications of one or more types of useful information. This data may be processed to generate one or multiple training datasets 236 for training of one or more machine learning models. In some embodiments, additional pertinent information may be provided, such as metadata (e.g., date and time of recordings), patient data (e.g., prescribed medications, medical history, etc.), survey data (e.g., answers to questions posed to the patient such as the time they last took one or more medications, whether they are experiencing any unusual circumstances that may alter their physical behaviors, etc.), or the like.

In one embodiment, generating one or more training datasets 236 includes generating various crops of video data. Temporal crops, spatial crops, temporal and spatial crops, etc., may be generated. In some embodiments, trained labelers may provide or assist in generation of crops. For example, a trained labeler may flag a short period of time where a movement indicative of a movement disorder has occurred, and the training dataset may include the short period of the recording as a labeled movement in the training dataset. As a further example, a trained labeler may input that a movement indicative of a movement disorder is present in a particular body part (e.g., the jaw), and a model may spatially crop at least a portion of the video to highlight the jaw when generating a training dataset.

To effectuate training, processing logic inputs the training dataset(s) 236 into one or more untrained machine learning models. Prior to inputting a first input into a machine learning model, the machine learning model may be initialized. Processing logic trains the untrained machine learning model(s) based on the training dataset(s) to generate one or more trained machine learning models that perform various operations as set forth above.

Training may be performed by inputting one or more of the movement disorder label data 210 and video data 212 into the machine learning model one at a time. In some embodiments, the training of the machine learning model includes tuning the model to receive video data 212 (e.g., potentially temporally and/or spatially cropped) and provide as output movement disorder label data 210. The machine learning model processes the input to generate an output. An artificial neural network includes an input layer that consists of values in a data point. The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the machine learning model can produce.

Accordingly, the output may include one or more predictions or inferences. For example, an output prediction or inference may include a prediction of a likelihood that a clip, video, or patient exhibits symptoms of a target movement disorder. The output may include a classification of a severity of the movement disorder. The output may include a description of the evolution through time of the movement disorder. The output may include predictions of future evolution of the movement disorder. The output may include a prediction of efficacy of treatment of the movement disorder. The output may include a recommendation, such as recommending additional screening for the patient.

Processing logic may compare the classification output of the model to the provided label and determine whether a threshold criterion is met (e.g., an accuracy of the model meets a threshold accuracy). Processing logic determines an error (i.e., a classification error) based on the differences between the processed output and training labels. Processing logic adjusts weights of one or more nodes in the machine learning model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons”, where each layer receives as input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the deep learning model. After one or more rounds of training, processing logic may determine whether a stopping criterion has been met. A stopping criterion may be a target level of accuracy, a target number of processed images from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria are met if accuracy of the machine learning model has stopped improving. If the stopping criterion has not been met, further training is performed. If the stopping criterion has been met, training may be complete. Once the machine learning model is trained, a reserved portion of the training dataset may be used to test the model.

As an example, in one embodiment, a machine learning model (e.g., movement disorder predictor 267) is trained to determine movement disorders (e.g., classification of one or more movement disorders). A similar process may be performed to train machine learning models to perform other tasks such as those set forth above. A set of many (e.g., thousands) combinations of labeled data may be collected and movement disorder classifications may be determined.

Once one or more trained machine learning models 238 are generated, they may be stored in model storage 245, and may be added to a movement disorder determination application. The movement disorder determination application may then use the one or more trained ML models 238 as well as additional processing logic to implement an automatic mode, in which user manual input of information is minimized or even eliminated in some instances. In some embodiments, a user (such as a health care provider) may instead or additionally choose video data to provide for analysis.

For model application workflow 217, according to one embodiment, input data 262 may be input into movement disorder predictor 267, which may include a trained neural network. Based on the input data 262, movement disorder predictor 267 outputs information indicating a predictive movement disorder determination as predictive movement disorder data 269. The movement disorder determination may include a classification of the movement disorder, a likelihood of the movement disorder, a severity of the movement disorder, etc.

In some embodiments, movement disorder predictor 267 may be a trained machine learning model that includes multiple trained machine learning models. For example, movement disorder predictor 267 may include various models directed toward generating predictions based on data segmentations. Movement disorder predictor 267 may include models trained to make predictions based on various spatial segmentations (e.g., various body parts). Movement disorder predictor 267 may include models trained to make predictions based on various data types. Movement disorder predictor 267 may include one or more models trained to make predictions based on video data (e.g., including audio and sequences of image data). Movement disorder predictor 267 may include one or more models trained to make predictions based on image data (e.g., sequences of images). Movement disorder predictor 267 may include one or more models trained to make predictions based on audio data (e.g., audio from one or more video recordings). Movement disorder predictor 267 may include one or more models directed toward generating a composite prediction based on output of several other models, e.g., movement disorder predictor 267 may be an ensemble model, a combination of models, or the like. In some embodiments, movement disorder predictor 267 may not be a combination of models. In some embodiments, movement disorder predictor 267 may make predictions based on video data input.

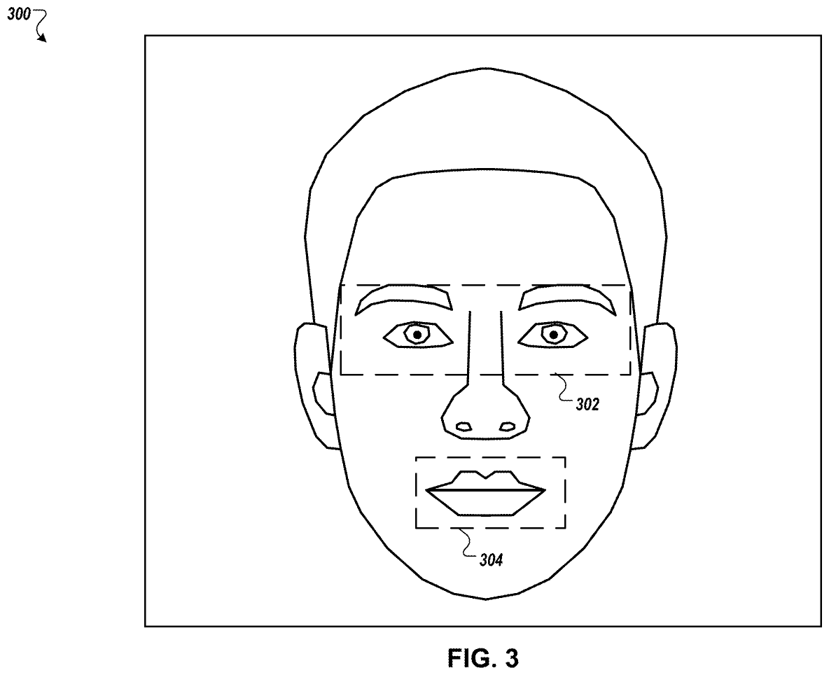

FIG. 3 depicts a frame of a video recording 300 as part of a video for use in determining a presence of a movement disorder, according to some embodiments. A video (e.g., video recording data including audio data and a series of image frames) may be provided for analysis to one or more machine learning models. A video may be recorded. The video may include a patients face, and may further include upper extremities, lower extremities, trunk, etc.

In some embodiments, a video may be segmented, e.g., temporally and/or spatially. Temporal segmentation may include generation of one or more video clips. For example, for generation of a labeled training dataset, a user may flag short portions of a recorded video that include movement disorder symptoms. In some embodiments, a user may label portions of a video based on observation of evidence of movement disorders. A user may provide a timestamp (e.g., via a graphical user interface of a client device) corresponding to evidence of a movement disorder. In some embodiments, a dataset used for training (including validating and testing operations) may include both temporal crops based on labels provided by a user (e.g., clips that include evidence of a movement disorder) and temporal crops that avoid portions labeled by a user (e.g., randomly selected clips that avoid evidence of a movement disorder). Providing both clips that include evidence of a movement disorder and clips that do not include evidence of a movement disorder from the same patient or recorded video may enable the machine learning model(s) to distinguish between various interfering factors that may be correlated with patients experiencing movement disorders and narrow in on actual movements, speech patterns, or other evidence of movement disorders.

Spatial segmentation may include cropping images to highlight a target body part. Spatial segmentation may include cropping images to include target portions of the patient's body. Video recording 300 includes a series of example spatial segmentations. Eyes crop 302 may be utilized to highlight involuntary movements of the eyes, eyelids, eye-adjacent muscles, brow, or the like. Lips crop 304 may be utilized to highlight involuntary movements of the lips, tongue, jaw, etc. Various other spatial segmentations of video data may be utilized. For example, one or more of jaw, lips, perioral, tongue, eyes, head, face musculature, hands, upper and/or lower extremities, and trunk crops may be utilized. Spatial crops may be provided to corresponding trained machine learning models, e.g., machine learning models configured to identify evidence of one or more movement disorders based on the target body part of the spatial crop.

FIGS. 4A-C are flow diagrams of methods 400A-C associated with training and utilizing machine learning models, according to certain embodiments. Methods 400A-C may be performed by processing logic that may include hardware (e.g., circuitry, dedicated logic, programmable logic, microcode, processing device, etc.), software (such as instructions run on a processing device, a general-purpose computer system, or a dedicated machine), firmware, microcode, or a combination thereof. In some embodiment, methods 400A-C may be performed, in part, by predictive system 110. Method 400A may be performed, in part, by predictive system 110 (e.g., server machine 170 and data set generator 172 of FIG. 1). Predictive system 110 may use method 400A to generate a data set to at least one of train, validate, or test a machine learning model, in accordance with embodiments of the disclosure. Methods 400B-C may be performed by predictive server 112 (e.g., predictive component 114), client device 120, and/or server machine 180 (e.g., training, validating, and testing operations may be performed by server machine 180). In some embodiments, a non-transitory machine-readable storage medium stores instructions that, when executed by a processing device (e.g., of predictive system 110, of server machine 180, of predictive server 112, etc.), cause the processing device to perform one or more of methods 400A-C.

For simplicity of explanation, methods 400A-C are depicted and described as a series of operations. However, operations in accordance with this disclosure can occur in various orders and/or concurrently and with other operations not presented and described herein. Furthermore, not all illustrated operations may be performed to implement methods 400A-C in accordance with the disclosed subject matter. In addition, those skilled in the art will understand and appreciate that methods 400A-C could alternatively be represented as a series of interrelated states via a state diagram or events.

FIG. 4A is a flow diagram of a method 400A for generating a data set for a machine learning model, according to some embodiments. Referring to FIG. 4A, in some embodiments, at block 401 the processing logic implementing method 400A initializes a training set T to an empty set.

At block 402, processing logic generates first data input (e.g., first training input, first validating input, first testing input) that may include one or more of video data, video crop data, N-dimensional embedding data associated with a video crop, or other data inputs described herein. In some embodiments, the first data input may include a first set of features for types of data and a second data input may include a second set of features for types of data. Input data may include historical data in some embodiments.

In some embodiments, at block 403, processing logic optionally generates a first target output for one or more of the data inputs (e.g., first data input). In some embodiments, the input includes one or more video clips and the output includes a determination of presence of evidence of movement disorder symptoms. In some embodiments, the input includes N-dimensional embeddings related to a series of video recordings of a patient, and the output includes a determination of the severity of symptoms of a movement disorder experienced by the patient. In some embodiments, the first target output is predictive data. Any of the functions of machine learning models described in connection with FIG. 2 (or combinations of functions, for example in the case of an ensemble machine learning model) may have associated data sets including corresponding inputs and target outputs. In some embodiments, no target output is generated (e.g., an unsupervised machine learning model capable of grouping or finding correlations in input data, rather than requiring target output to be provided).

At block 404, processing logic optionally generates mapping data that is indicative of an input/output mapping. The input/output mapping (or mapping data) may refer to the data input (e.g., one or more of the data inputs described herein), the target output for the data input, and an association between the data input(s) and the target output. In some embodiments, such as in association with machine learning models where no target output is provided, block 404 may not be executed.

At block 405, processing logic adds the mapping data generated at block 404 to data set T, in some embodiments.

At block 406, processing logic branches based on whether data set T is sufficient for at least one of training, validating, and/or testing a machine learning model, such as model 190 of FIG. 1. If so, execution proceeds to block 407, otherwise, execution continues back at block 402. It should be noted that in some embodiments, the sufficiency of data set T may be determined based simply on the number of inputs, mapped in some embodiments to outputs, in the data set, while in some other embodiments, the sufficiency of data set T may be determined based on one or more other criteria (e.g., a measure of diversity of the data examples, accuracy, etc.) in addition to, or instead of, the number of inputs.

At block 407, processing logic provides data set T (e.g., to server machine 180) to train, validate, and/or test machine learning model 190. In some embodiments, data set T is a training set and is provided to training engine 182 of server machine 180 to perform the training. In some embodiments, data set T is a validation set and is provided to validation engine 184 of server machine 180 to perform the validating. In some embodiments, data set T is a testing set and is provided to testing engine 186 of server machine 180 to perform the testing. In the case of a neural network, for example, input values of a given input/output mapping (e.g., numerical values associated with data inputs 210A) are input to the neural network, and output values (e.g., numerical values associated with target outputs) of the input/output mapping are stored in the output nodes of the neural network. The connection weights in the neural network are then adjusted in accordance with a learning algorithm (e.g., back propagation, etc.), and the procedure is repeated for the other input/output mappings in data set T. After block 407, a model (e.g., model 190) can be at least one of trained using training engine 182 of server machine 180, validated using validating engine 184 of server machine 180, or tested using testing engine 186 of server machine 180. The trained model may be implemented by predictive component 114 (of predictive server 112) to generate predictive data 168 for generating predictive data and/or for performing a corrective action associated with a movement disorder.

FIG. 4B is a flow diagram of a method 400B for utilizing machine learning models in determining whether evidence of movement disorders is captured, according to some embodiments. At block 410, processing logic obtains video data of a patient, comprising image data and audio data. In some embodiments, only image data may be utilized in movement disorder determinations. In some embodiments, only audio data may be utilized in movement disorder determinations. In some embodiments, image data (e.g., a series of image frames) and audio data may be provided to the same set of machine learning models, separate sets of machine learning models, or overlapping sets of machine learning models.

In some embodiments, the video data may be or include a recording of the patient. The video data may include video recording data of the patient. In some embodiments, the video data may be a recording of a meeting between the patient and a healthcare provider. In some embodiments, the video data may be or include a recording of the patient responding to prompts, e.g., movement disorder screening prompts potentially including questions to answer, motions or positions to perform, and the like. The instructions may be provided by a healthcare provider, a screener, or automatically by the screening system, such as by on-screen text instructions, recorded and/or generated audio instructions (e.g., text-to-speech generation), or the like. In some embodiments, the video data may be a recording of a meeting between the patient and a healthcare provider during which the patient is prompted to perform actions that may be used for making a determination in connection with a movement disorder. For example, the patient may be asked questions, prompted to perform certain actions or activate certain muscles, or the like, that may display evidence of a movement disorder.

In some embodiments, the video data may be preprocessed. For example, the video data may be segmented. The video data may be segmented spatially and/or temporally. In some embodiments, the video data may be cropped temporally (e.g., split into one or more shorter duration clips) and/or spatially (e.g., separated into components highlighting target body parts or target portions of a body of the patient). In some embodiments, various segments may be provided to machine learning models for analysis. For example, a number of temporal crops, a number of spatial crops, and an entire recording may all be provided to one or more machine learning models for analysis. In some embodiments, spatial crops may include crops highlighting jaw, lips, perioral, tongue, eyes, head, face muscles, hands, upper and/or lower extremities, trunk, or the like, of the patient. In some embodiments, temporal crops may include clips of about one second in duration, crops between 0.5 and 5 seconds, clips between 0.1 and 10 seconds, or other lengths of clip. In some embodiments, length of a clip may approximately correspond to an expected duration of a indication of a movement disorder (e.g., a duration of an involuntary movement typical to one or more target movement disorders).

At block 412, processing logic provides the video data to a first trained machine learning model. The first trained machine learning model may be an ensemble model. The first trained machine learning model may be configured to receive the video data and provide a determination in association with one or more movement disorders. The first trained machine learning model may include a number of other models (e.g., an ensemble machine learning model). The first trained machine learning model may include one or more models for image data, one or more models for audio data, one or more models for different spatial crops (e.g., highlighting different body parts), etc.

In some embodiments, the first trained machine learning model may include several models (e.g., may be an ensemble model) that act in sequence on one or more sets of video data. For example, a first model may receive a crop of the video data, and generate an indication of a likelihood that a target movement disorder was exhibited in the crop. A second model may receive indications of a likelihood that the target movement disorder was exhibited in one or more crops from the first model, and generate as output a composite indication of a likelihood that the target movement disorder was exhibited in the video comprising several analyzed clips. A third model may receive data related to several video recordings, and generate as output a composite indication of a determination associated with a patient based on several video recordings of the patient. For example, the third model may generate a composite indication of movement disorder progression, movement disorder severity, or the like.

At block 414, processing logic obtains output from the first trained machine learning model based on the video data. The output includes an indication that the patient exhibits symptoms of one or more target movement disorders in association with the video data. The target movement disorder may be tardive dyskinesia. The target movement disorder may be Parkinson's disease. The target movement disorder may be Huntington's chorea. The target movement disorder may be essential tremor, dystonia, Tourette syndrome, restless leg syndrome, or another movement disorder that may be exhibited via image and/or audio data.

At block 416, processing logic provides an alert to a user indicative of the one or more target movement disorders. In some embodiments, the alert may be provided to a healthcare provider. In some embodiments, the alert may be indicative of whether or not evidence of a movement disorder was detected. In some embodiments, the alert may be indicative of a likelihood that evidence of a movement disorder is included in the video data. In some embodiments, the alert may be indicative of a severity or progress of the movement disorder. In some embodiments, the alert may be provided via a graphical user interface of a client device. In some embodiments, the alert may prompt a user to perform additional actions, such as performing additional screening or diagnostic techniques.

FIG. 4C is a flow diagram of a method 400C for training a machine learning model for making determinations in association with movement disorders, according to some embodiments. At block 420, processing logic obtains a first plurality of video data of a first plurality of patients. The video data may include recorded image data (e.g., a series of image frames) and audio data. The patients may include both patients that are experiencing movement disorder symptoms and patients that are not experiencing movement disorder symptoms.

At block 422, processing logic performs cropping of each of the first plurality of video data to generate a first plurality of video data crops. In some embodiments, the cropping may be temporal, e.g., separating video data into clips of a target duration or range of durations. In some embodiments, the cropping may be spatial, e.g., to highlight one or more target body parts and/or portions of the body of the patients. In some embodiments, cropping may be both temporal and spatial. In some embodiments, cropping may be facilitated by one or more users. For example, a trained user may review a recording of a patient. The user may flag portions of the video that include demonstrations of a movement disorder. Processing logic may generate clips based on the flagged portions of the recording. Processing logic may generate both clips that include involuntary movement and clips that do not include involuntary movement from the same recording of the same patient. Providing clips of the same patient that both include and do not include evidence of a movement disorder may enable the machine learning model to distinguish between natural movements and movements associated with a movement disorder.

At block 424, processing logic receives a first plurality of labels associated with each of the first plurality of video data crops. The first plurality of labels comprises an indication of a presence or absence of evidence of a movement disorder in the first plurality of video data crops. The labels may be generated by one or more users, e.g., users trained to recognize movement disorders.

At block 426, processing logic trains a first machine learning model by providing the first plurality of video data crops as training input and the first plurality of labels as target output. The first machine learning model is configured to generate output indicative of whether an input video data crop includes an indication of the movement disorder.

Machine learning models configured to perform different tasks may be trained in an analogous method, utilizing training data targeted to the intended usage of the machine learning model. For example, a machine learning model configured to receive indications of movement disorders from one or more clips of a video recording and provide a composite indication related to a determined likelihood of evidence of a movement disorder in the clip as a whole may be provided as training input indications of movement disorders from training clips and labels associated with the video recordings of the training clips as target output. As a further example, a model may be configured to provide a composite indication of a determination of whether a patient is experiencing symptoms based on data from a series of video recordings. The model may be trained based on labels indicating a severity of a movement disorder of a patient as target output, and indications based on a series of recordings as training input.

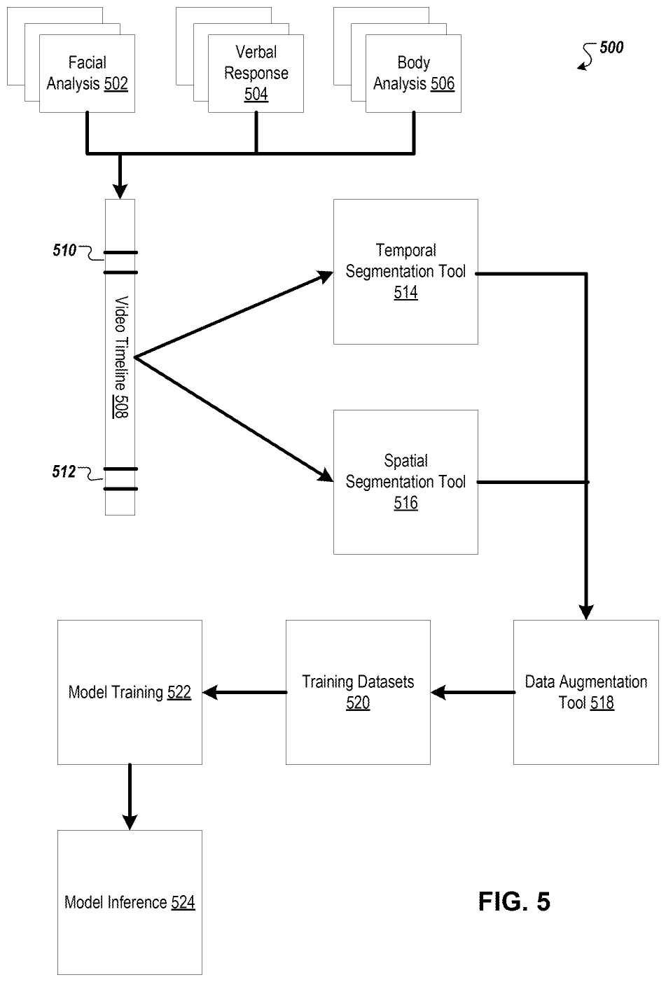

FIG. 5 depicts an operation flow 500 for making a determination in association with a movement disorder, according to some embodiments. In order to generate data both for training and inference, video data of a patient is generated. In some embodiments, video data may be a recording of a virtual or in-person meeting with a healthcare provider. In some embodiments, a series of targeted questions and/or instructions may be provided to the patient that are used to determine whether a movement disorder is present. In one example, questions are asked and/or instructions given to the patient that target facial responses, e.g., responses are recorded for facial analysis 502. Further instructions may be provided to the patient to target other symptomatic sites for movement disorders, such as gauging verbal response 504 and body analysis 506. Questions may be similar to questions asked during a conventional movement disorder screening, such as an AIMS test administered to determine whether a patient is experiencing tardive dyskinesia symptoms. A patient may be asked questions, as well as asked to perform actions such as open their mouth, tap fingers, stare into the camera, etc.

A video timeline 508 is generated based on recording the patient. In practice, many patients may be recorded, and multiple recordings of each patient may be made. Portions of video timeline 508 may be labeled. For example, a user trained to recognize movement disorders may highlight portions of the video (e.g., portions 510 and 512) where evidence of a target movement disorder are present. The user may further input a description of a body part where the movement disorder is demonstrated.

The labeled video timeline 508 is then provided to temporal segmentation tool 514 and spatial segmentation tool 516. The segmentation tools utilize the labels provided by the user(s) to segment the video into segments. Segmentation may be spatial, e.g., based on body part labels provided by the user. Segmentation may be temporal, e.g., based on timestamps flagging for movement disorders provided by the user. In some embodiments, segments may be generated that are segmented both spatially and temporally, e.g., a one-second clip cropped to focus on a target body part. Segmentation may be guided by user labels. Segmentation may include separating portions (temporally and/or spatially) that both do and do not provide evidence of movement disorders. Providing both segments that do and do not exhibit movement disorders may assist with training a reliable model for distinguishing movement disorders.

Segments are provided to data augmentation tool 518. Data augmentation tool may provide some variations to the data clips. Variations may be used to protect the model against over-fitting based on erroneous data correlations. For example, video segments may be adjusted in color, brightness, contrast, clarity, size, frame rate, aspect ratio, rotation, etc. Such variations may effectively increase an available size of the training data, as well as cause the trained model to be more robust to variations in input data.

Augmented data is used to generate training datasets 520 (e.g., as described in connection with FIGS. 2 and 4A). Data flow further proceeds to model training 522 (e.g., as described in connection with FIGS. 2 and 4C) and model inference 524 (e.g., as described in connection with FIGS. 2 and 4B).

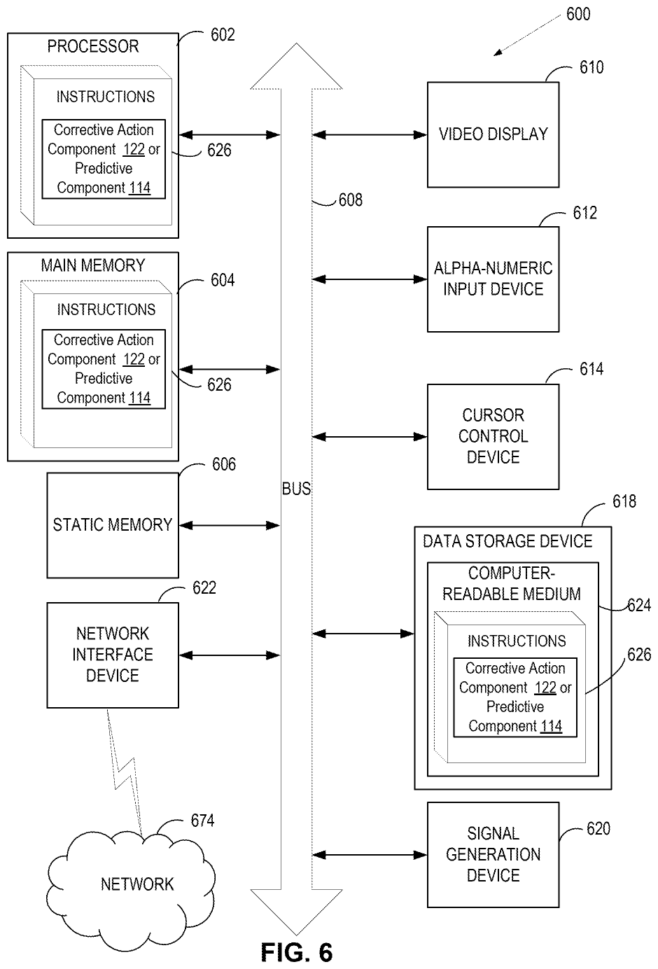

FIG. 6 is a block diagram illustrating a computer system 600, according to some embodiments. In some embodiments, computer system 600 may be connected (e.g., via a network, such as a Local Area Network (LAN), an intranet, an extranet, or the Internet) to other computer systems. Computer system 600 may operate in the capacity of a server or a client computer in a client-server environment, or as a peer computer in a peer-to-peer or distributed network environment. Computer system 600 may be provided by a personal computer (PC), a tablet PC, a Set-Top Box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 600 may include a processing device 602, a volatile memory 604 (e.g., Random Access Memory (RAM)), a non-volatile memory 606 (e.g., Read-Only Memory (ROM) or Electrically-Erasable Programmable ROM (EEPROM)), and a data storage device 618, which may communicate with each other via a bus 608.

Processing device 602 may be provided by one or more processors such as a general purpose processor (such as, for example, a Complex Instruction Set Computing (CISC) microprocessor, a Reduced Instruction Set Computing (RISC) microprocessor, a Very Long Instruction Word (VLIW) microprocessor, a microprocessor implementing other types of instruction sets, or a microprocessor implementing a combination of types of instruction sets) or a specialized processor (such as, for example, an Application Specific Integrated Circuit (ASIC), a Field Programmable Gate Array (FPGA), a Digital Signal Processor (DSP), or a network processor).

Computer system 600 may further include a network interface device 622 (e.g., coupled to network 674). Computer system 600 also may include a video display unit 610 (e.g., an LCD), an alphanumeric input device 612 (e.g., a keyboard), a cursor control device 614 (e.g., a mouse), and a signal generation device 620.

In some embodiments, data storage device 618 may include a non-transitory computer-readable storage medium 624 (e.g., non-transitory machine-readable medium) on which may store instructions 626 encoding any one or more of the methods or functions described herein, including instructions encoding components of FIG. 1 (e.g., predictive component 114, corrective action component 122, model 190, etc.) and for implementing methods described herein.

Instructions 626 may also reside, completely or partially, within volatile memory 604 and/or within processing device 602 during execution thereof by computer system 600, hence, volatile memory 604 and processing device 602 may also constitute machine-readable storage media.

While computer-readable storage medium 624 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and computer program components, or in computer programs.

Unless specifically stated otherwise, terms such as “receiving,” “performing,” “providing,” “obtaining,” “causing,” “accessing,” “determining,” “adding,” “using,” “training,” “reducing,” “generating,” “correcting,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. Also, the terms “first,” “second,” “third,” “fourth,” etc. as used herein are meant as labels to distinguish among different elements and may not have an ordinal meaning according to their numerical designation.

Examples described herein also relate to an apparatus for performing the methods described herein. This apparatus may be specially constructed for performing the methods described herein, or it may include a general purpose computer system selectively programmed by a computer program stored in the computer system. Such a computer program may be stored in a computer-readable tangible storage medium.

The methods and illustrative examples described herein are not inherently related to any particular computer or other apparatus. Various general purpose systems may be used in accordance with the teachings described herein, or it may prove convenient to construct more specialized apparatus to perform methods described herein and/or each of their individual functions, routines, subroutines, or operations. Examples of the structure for a variety of these systems are set forth in the description above.

The above description is intended to be illustrative, and not restrictive. Although the present disclosure has been described with references to specific illustrative examples and embodiments, it will be recognized that the present disclosure is not limited to the examples and embodiments described. The scope of the disclosure should be determined with reference to the following claims, along with the full scope of equivalents to which the claims are entitled.

What is claimed is:

1. A method, comprising:
obtaining, by a processing device, video data of a patient, comprising image data and audio data;
providing, by the processing device, the video data to a first trained machine learning model;
obtaining output from the first trained machine learning model based on the video data, wherein the output comprises a first indication that the patient exhibits symptoms of one or more target movement disorders in association with the video data; and
providing an alert to a user indicative of the one or more target movement disorders.

2. The method of claim 1, further comprising:
obtaining recording data of the patient; and
generating a first video crop of the recording data by performing temporal cropping of the recording data, wherein the video data of the patient comprises the recording data and the first video crop of the recording data.

3. The method of claim 2, further comprising generating a plurality of video crops of the recording data, wherein each of the plurality of video crops is between 0.1 seconds and 10 seconds in length, and wherein the video data of the patient further comprises the plurality of video crops.

4. The method of claim 1, further comprising:
obtaining recording data of the patient; and
generating a first video crop of the recording data by performing spatial cropping of the recording data, wherein the video data of the patient comprises the recording data and the first video crop of the recording data.

5. The method of claim 4, further comprising generating a plurality of video crops of the recording data, wherein each of the plurality of video crops comprises a spatial portion of the recording data, comprising a target portion of a body of the patient, and wherein the video data of the patient further comprises the plurality of video crops.

6. The method of claim 1, wherein the first trained machine learning model comprises a first model configured to receive as input the image data, and a second model configured to receive as input the audio data.

7. The method of claim 1, wherein the first trained machine learning model comprises:
a second trained machine learning model, wherein the second trained machine learning model is configured to receive as input a crop of the video data of the patient, and generate as output a second indication of a likelihood that a first target movement disorder was exhibited in the crop of the video data of the patient; and
a third trained machine learning model, wherein the third trained machine learning model is configured to receive as input one or more indications of a likelihood that the first target movement disorder was exhibited in one or more crops of the video data of the patient, and generate as output a composite indication of a likelihood that the first target movement disorder was exhibited in the video data of the patient.

8. The method of claim 1, wherein the first trained machine learning model comprises:
a second trained machine learning model, wherein the second trained machine learning model is configured to generate as output a second indication of a likelihood that a first target movement disorder of the one or more target movement disorders was exhibited in the video data of the patient; and
a third trained machine learning model, wherein the third trained machine learning model is configured to receive as input the second indication of a likelihood that the first target movement disorder was exhibited in the video data of the patient, and a plurality of indications of likelihood that the first target movement disorder was exhibited in a plurality of video data of the patient, and wherein the third trained machine learning model is configured to generate as output a third indication of a severity of symptoms of the patient in association with the first target movement disorder.

9. The method of claim 1, wherein a first target movement disorder of the one or more target movement disorders comprises one of:
tardive dyskinesia;
Huntington's chorea; or
Parkinson's disease.

10. A method, comprising:
obtaining, by a processing device, a first plurality of video data of a first plurality of patients;
performing cropping of each of the first plurality of video data to generate a first plurality of video data crops;
receiving a first plurality of labels associated with each of the first plurality of video data crops, wherein the first plurality of labels comprises a first indication of a presence or absence of evidence of a movement disorder in the first plurality of video data crops; and
training a first machine learning model by providing the first plurality of video data crops as training input and the first plurality of labels as target output, wherein the first machine learning model is configured to generate output indicative of whether an input video data crop comprises an indication of the movement disorder.

11. The method of claim 10, further comprising:
receiving a second plurality of labels, wherein each label of the second plurality of labels comprises a second indication of a presence or absence of evidence of the movement disorder in the first plurality of video data; and
training a second machine learning model by providing output of the first machine learning model as training input and the second plurality of labels as target output, wherein the second machine learning model is configured to generate output indicative of whether a video comprising the input video data crop comprises a third indication of the movement disorder.

12. The method of claim 11, further comprising:
receiving a third plurality of labels, wherein each label of the third plurality of labels comprises a fourth indication of a severity of the movement disorder in an associated patient; and
training a third machine learning model by providing output from the second machine learning model as training input and providing the third plurality of labels as target output, wherein the third machine learning model is configured to generate output indicative of a prediction of a severity of movement disorder symptoms of a patient based on one or more videos of the patient.

13. The method of claim 10, wherein the first plurality of video data crops comprise one or more of:
temporal crops; or
spatial crops, wherein each spatial crop includes a target portion of a patient's body.

14. A non-transitory machine-readable storage medium, storing instructions which, when executed, cause a processing device to perform operations comprising:
obtaining video data of a patient, comprising image data and audio data;
providing the video data to a first trained machine learning model;
obtaining output from the first trained machine learning model based on the video data, wherein the output comprises a first indication that the patient exhibits symptoms of one or more target movement disorders in association with the video data; and
providing an alert to a user indicative of the one or more target movement disorders.

15. The non-transitory machine-readable storage medium of claim 14, wherein the operations further comprise:
obtaining recording data of the patient; and
generating a first video crop of the recording data by performing temporal cropping of the recording data, wherein the video data of the patient comprises the recording data and the first video crop of the recording data.

16. The non-transitory machine-readable storage medium of claim 14, wherein the operations further comprise:
obtaining recording data of the patient; and
generating a first video crop of the recording data by performing spatial cropping of the recording data, wherein the video data of the patient comprises the recording data and the first video crop of the recording data.

17. The non-transitory machine-readable storage medium of claim 16, wherein the operations further comprise generating a plurality of video crops of the recording data, wherein each of the plurality of video crops comprises a spatial portion of the recording data, comprising a target portion of a body of the patient, and wherein the video data of the patient further comprises the plurality of video crops.

18. The non-transitory machine-readable storage medium of claim 14, wherein the first trained machine learning model comprises:
a second trained machine learning model, wherein the second trained machine learning model is configured to receive as input a crop of the video data of the patient, and generate as output a second indication of a likelihood that a first target movement disorder of the one or more target movement disorders was exhibited in the crop of the video data of the patient; and
a third trained machine learning model, wherein the third trained machine learning model is configured to receive as input one or more indications of a likelihood that the first target movement disorder was exhibited in one or more crops of the video data of the patient, and generate as output a composite indication of a likelihood that the first target movement disorder was exhibited in the video data of the patient.

19. The non-transitory machine-readable storage medium of claim 14, wherein the first trained machine learning model comprises:
a second trained machine learning model, wherein the second trained machine learning model is configured to generate as output a second indication of a likelihood that a first target movement disorder of the one or more movement disorders was exhibited in the video data of the patient; and
a third trained machine learning model, wherein the third trained machine learning model is configured to receive as input the second indication of a likelihood that the first target movement disorder was exhibited in the video data of the patient, and a plurality of indications of likelihood that the first target movement disorder was exhibited in a plurality of video data of the patient, and wherein the third trained machine learning model is configured to generate as output a third indication of a severity of symptoms of the patient in association with the target movement disorder.

20. The non-transitory machine-readable storage medium of claim 14, wherein a target movement disorder of the one or more target movement disorders comprises one or more of:
tardive dyskinesia;
Huntington's chorea; or
Parkinson's disease.
